(19) United States
(12) Patent Application Publication
(10) Pub. No.: US 2014/0005268 A1
(43) Pub. Date: Jan. 2, 2014

(54) PHARMACEUTICAL COMPOSITION FOR TREATING OXIDATIVE STRESS-INDUCED PATHOLOGY AND USE THEREOF

(71) Applicant: Eduardo Motta Alves Peixoto, Sao Paulo (BR)

(72) Inventor: Eduardo Motta Alves Peixoto, Sao Paulo (BR)

(21) Appl. No.: 13/980,606

(22) PCT Filed: Jan. 18, 2012
(86) PCT No.: PCT/BR2012/000008

§ 371 Date: Sep. 20, 2013

(30) Foreign Application Priority Data
Jan. 19, 2011 (BR) PI1100663-3

(51) Int. Cl. A61K 31/198

(52) U.S. Cl. CPC A61K 31/198
USPC 514/562

(57) ABSTRACT

Pharmaceutical composition for treating oxidative stress induced pathology, comprising an association of the active ingredients methionine, cysteine and phenylalanine in association with at least one pharmaceutical excipient; said active ingredients being present in a methionine/phenylalanine molar ratio between 2.5 and 12.5; and in a methionine/cysteine molar ratio between 2 and 10.5; and the three active ingredients being present in free state, such as inner salt, or in the form of pharmaceutically acceptable salt; wherein said pharmaceutical composition is used for treating one of the oxidative stress induced pathologies, including intestinal system disorders, such as intestinal wall irritations, hemorrhoids of types 1, 2, 3 and 4 of Goligher classification. It is also disclosed the use of said composition in preparing a medication for treating oxidative stress induced pathology.

### FIELD OF THE INVENTION

The present invention relates in general to treatment of hemorrhoids and, in particular, aims at a new association of active ingredients with antioxidant activity of sulfur-containing amino acid residues in proteins.

### BACKGROUND OF INVENTION

For centuries, hemorrhoids have been treated by surgeons. Therapies for hemorrhoid topical treatment date from 1700 BC in old Egypt papyri. One of the first surgical treatments (Kaidar-Person et al., J. Am. Coll. Surg., 2007, 204, 102-117) was described in the Hippocratic Oath, dating from 460 BC and it suggests transfixing them with a needle and tying them up with a large thick woolen yarn (Parks, Guys Hosp. Rep., 1955, 104,135-156). After so many centuries, the accurate etiology thereof is still obscure and a definitive treatment is yet to be defined (Hardy et al., Dig. Surg.,2005, 22,26-33). Nowadays, hemorrhoids are estimated to affect between 4.4 and 36% of general population (Loder et al., Br. J. Surg., 1994, 81, 946-954).

Reactive Oxygen Species.—In recent years, a series of studies has increasingly shown the role of Reactive Oxygen Species (ROS). Different definitions of what such species would be, free radicals, oxidants, etc., such as superoxide anion (O²) and hydrogen peroxide (H₂O₂), may be found in the literature (Halliwell, B. & Gutteridge, Free

Radical in Biology and Medicine, Ox. Univ. Press, 1999). They may be generated as part of the aerobic and normal existence of the cell. Thus, in turn, these reactive species promote the production of many other molecules capable of inducing oxidative stress in the cells. Usually, in the organism, there is a series of processes and mechanisms capable of globally maintaining and compensating this delicate balance between the production and elimination of these oxidants. For such, various enzymatic and non-enzymatic processes are used. However, each day there is additional evidence that the cells may use small and/or local changes in the concentrations of oxidants/ROS as signs of transduction of cell signs (Suzuki et al., Free Rad. Biol. Med., 1997, 22, 269-285; Fukagawa, Proc. Soc. Exp. Biol. Med., 1999, 222, 293-298; Finkel, FEBS Letters, 2000, 476, 52-54).

Many ROS are actually free radicals having non-paired electrons which many times are not very reactive and have too short a half-life. An example, starting with the superoxide, is Fenton's reaction, which produces H₂O₂ and then the hydroxyl radical (OH⁻.) using transition metal ions, such as Fe^{3+/}Fe²⁺. It should be considered that the hydroxyl radical is the one which is in fact envolved in oxidatively destroying or damaging various bimolecular species. On the other hand, even though they are very reactive, it should be expected that these reactive radicals have very low diffusion coefficients. Thus, it limitates the action radius thereof. The hydroxyl radical has an average life of only 2 ns and the diffusion radius thereof is only 2 nm (Haugland, Handbook of Fluorescent Probes and Res. Chem. Molecular probes, Eugenes, Oreg., USA, 1996). On the other hand, other less reactive species may have a long distance effect not only inside a cell, but even between two cells. For example, the NO radical, a weak radical, i.e., less reactive, works as a messenger not only inside the cells but also between two cells (Jaffrey & Snyder, Ann. Rev. Cell Dev. Bio., 1995, 11, 417-440; Yun et al., Critical Rev. Neurobiol., 1996, 10, 291-316; Squadrito & Pryor, Free Radical Biol. Med., 1, 1998, 25, 392-403).

ROS, specially in the presence of other cofactors such as metal ions, are capable of, through an oxidation process, modifying a series of cell components (Halliwell & Gutteridge, Free Radical in Biology and Medicine, Ox. Univ. Press, 1999). The oxidative stress induced by these reactive agents may lead to a lipid peroxidation. DNA damage and poly ADP-ribose synthetase activation, besides causing damages to proteins and carbohydrates. For example, lipid peroxidation may result in the oxidation of cholesterol and fatty acids, and may compromise the intregrity of the cell membrane; damaging the DNA may eventually lead to cell death or to the abnormal growth thereof. The damages mediated by oxidations may thus damage the DNA, lipids and proteins and thereby contribute to aging, organic changes associated to age and many degenerative diseases also associated to age (Stadtman & Berlett, Drug Metab. Rev., 1998, 30,225-243).

Amino Acid Oxidation.—One of the main targets of ROS and cell oxidants is the amino acid residues in proteins in our organism. All the amino acids may be oxidized. Among these oxidative modifications, polypeptide backbone is included as well as the side chains of a molecule formed by amino acid parts. The oxidative abstraction of a hydrogen atom of the carbon atom leads to peptide bond breakage (Stadtman & Berlett, Reactive Oxygen-Mediated Protein Oxidation in Aging and disease, Kluwer Acad. Plenum, New York, 1999). The amino acid side chains of a protein are easily oxidized and, thus, this amino acid's properties are altered, and may thereby potentially cause a modification which alters the properties and functions of the protein modified in this fashion (Stadtman & Berlett, Drug Metab. Rev., 1998, 30, 225-243; Halliwell & Gutteridge, Free Radical in Biology and Medicine, Ox. Univ. Press, 1999; Stadtman & Berlett, Reactive Oxygen-Mediated Protein Oxidation in Aging and Disease, Kluwer Acad. Plenum, New York, 1999). As it should be expected, different amino acids are more or less easily oxidized. Sulfur-containing amino acids, S, such as cysteine and methionine, are specially sensitive to reactions mediated by ROS. However, the arginine, lysine, proline, histidine, tryptophan and tyrosine side chains are also known for the capacity of being modified in oxidative processes (Berlett& Stadtman, J. Biol. Chem., 1997, 272, 20313-20316; Stadtman & Berlett, 1999). The extension of the alteration endured by a protein is measured by the protein's carbonyl content (Levine et al., Methods in Enzymology, 1994, 233, 346-357); this method, however, has some questionable aspects (Halliwell & Gutteridge, 1999). It is important to highlight that an expressive moiety of the total proteins of a human being may be oxidized (Stadtman. Science, 1992, 257, 1220-1224). This way, it should be expected that a series of health conditions may occur in a human being, which are mostly associated to age, caused by tissue breakdown due to the mentioned causes.

Sulfur-Containing Amino Acids: cysteine and methionine.—Both the cysteine and methionine molecule possess highly reactive sulfur in their side chains and it presents a preferable target for ROS's. It is known, however, from a physiological point of view, that the oxidation of residues both from cysteine and methionine in proteins may be reversible.

The cysteine oxidation containing a thiol group, S—H, is sometimes promoted by the presence of metal ion traces, such as Cu²⁺, Fe²⁺, Co²⁺ and Mn²⁺, providing a variety of products including those having sulfinic ion, disulfide and sulfonic ions (Finkel, FEBS Letters, 2000, 476, 52-54). Physiologically, forming disulfide may be the most likely consequence of cysteine oxidation (Creighton, Proteins Structure and Mol. Properties, Freeman, N.Y., 1993). Disulfides may be easily reduced to thiols by using glutathione in vivo or dithiothreitol (DTT) in vitro. The reversion of cysteine oxidation has been considered an important cell redox sensor in some proteins (Finkel, FEBS Letters, 2000, 476, 52-54). Experimentally, stronger oxidants may produce sulfonic acids and eventually sulfinic acid. The modulation of N-type inactivation of potassium channels depending on the voltage is an example of a fast and reversible phenomenum mediated by the oxidation of a specific residue of cysteine in the amino terminal of the channel protein (Kv1.4) (Ruppersberg et al., Nature,1991,352, 711-714) or to the associated subunit (Rettig et al., Nature,1994, 369, 289-294; Heinemann et al. FEBS Letters, 1995, 377, 383-389).

Methionine Oxidation.—Methionine is oxidized to methionine sulfoxide (MetO), MeSOX, MetSO or MsX) by adding an extra oxygen atom. The presence of methionine sulfoxide has been documented in proteins (e.g. Gao et al., Biophys. J., 1998, 74, 1115-1134), indicating that the oxidation of a methionine side chain is a relevant physiological phenomena. For these reasons, changes of this type (oxidation), which may occur in methionine residues in proteins of our organism are very important and thus, it should be expected that this alters the function of the protein of interest. Under normal conditions, the methionine side chain is long, flexible and non-polar (Richardson & Richardson, Principles and Patterns of Protein Conformation, Plenum Press, NY, 1989, pag. 1-98). Even though we have observed that, in some cases, methionine residues are not in the external surface of a protein molecule, other proteins have multiple exposed residue (Levine et al. Proc. Nat. Acad. Sci. USA, 1996, 93, 15036-15040). The methionine sulfoxide side chain having an extra oxygen atom is rigid and more polar than that of methionine and, thus, they are different from each other. Furthermore, the hydrophobicity index of the methionine sulfoxide has been estimated as being similar to that of lysine, a positively charged amino acid (Black & Mould, Analytical Biochem., 1991, 193,72-82). Thus, considering only the hydrophobicity aspect, this is equivalent to the substitution of methionine by another amino acid having a charge. It should be noticed that merely the charge does not explain all the functional changes caused by methionine oxidation (protein) (Yin et al., Biochem., 1999, 38, 13654-13660). Thus, it should be considered that oxidation and specific reduction of methionine residues in proteins result in substantial consequences for the protein functions and may constitute a mechanism for protein regulation. This way, it has been suggested that the extension of the cell oxidation is underestimated in part because the amino acid analysis based on acid digestion is not suitable for detecting methionine sulfoxide (Squier & Bigelow, Frontiers in Bioscience, 2000, 5, D504-526).

Methionine side chains may be oxidized in the organism, by a series of different ROS, such as O²⁻, H²O², peroxonitrite (ONOO—), or .OH. Experimentally, there are many aspects which make it more difficult to reproduce these oxidative processes in vitro, since most of them do not reproduce in satisfactory fashion what happens in the organism. Thus, for example, many conflicting results may be explained by the presence of different concentrations of contaminating ions.

The changes in the physical and chemical properties associated to methionine oxidation mentioned above, make it clear that they induce serious alterations in the protein functions and considerably alter the cell physiology and consequently, human physiology, as it should be observed in certain intestinal disfunctions where the walls of certain vessels become fragile or easily irritable. On the other hand, there is evidence that there are proteins having methionine residues and that, however, do not appear to have their functions altered. This way, it may be speculated the existence of a cyclic mechanism for oxidation/reduction of such methionine residues as being endogenous and having local antioxidant capacity. In this case, vessels from the intestine wall may represent an example. Thus, the local alteration of this balance may lead to intestine diseases characterized by the fragility of local vessels or membranes, ranging from a simple constipation to a membrane fraying with local bleeding, whereas the balance may also be reestablished leading to the reversion of this scenario with the presence of suitable amino acid(s).

Methionine Sulfoxide Reductase (MSRA).—As in the case of oxidized cysteine, MetO may be physiologically reduced to methionine. The reduction reaction of MetO to methionine is catalysed by the peptide enzyme methionine sulfoxide reductase (MSRA) using tioredoxin in vivo or DTT in vitro (Moskovitz et al. Proc. Nat. Acad. Sci. USA, 1996, 93, 2095-2099). A recent study showed that MSRA preferably reduces L-methionine sulfoxide (Sharov et al., FEBS Letters, 1999, 455, 247-250). MSRA is a relatively small enzyme that may be found in a great variety of organisms, ranging from bacteria (Moskovitz et al. J. Bacteriology, 1995, 177, 502-507) to plants (Sadanandom et al., Plant Phys., 2000, 123, 255-264) and mammals (Moskovitz et al., Proc. Nat. Acad. Sci. USA, 1996, 93, 2095-2099), including human beings (Kuschel et al., FEBS Letters, 1999, 456, 17-21).

MSRA is found expressed in different manners in different tissues indicating that the enzyme must have specific physiological functions.

### DESCRIPTION OF THE INVENTION

Surprisingly, the association of methionine and other amino acids essential of the invention is revealed to be provided with a very special activity in restoring certain tissues of human organism. This could be appreciated in the fast recovery of the intestine wall of humans anally treated. This effect is most likely characterized by the antioxidant activity of various of the ingredients thereof, and specially that of free methionine avoiding the oxidation of methionine residues in proteins of human tissue more easily reacting with the different ROS's present there.

The associations, according to the invention, do not increase the hemorrhagic risk appreciated over the elongation of the bleeding time, which, generally speaking, when initially existent, stops in 3 to 4 days by anally using this invention and, on the other hand, they have extremely low toxicity. As the associations of the present invention are comprised of associations of amino acids essential to human beings, their toxicity is not only compatible to its usage as a medication for treating disorders and diseases associated to aging of the intestinal tissue as well as to those attributed to oxidative stress of the tissue for excessive ROS's or perhaps local MSRA deficiency.

The associations, according to the invention, may be formulated in pharmaceutical compositions for administering to human beings, for treating the abovementioned diseases and metabolic disfunctions.

According to the invention, methionine and other associated amino acids may be administered in pure form, such as inner salt, or in the form of pharmaceutically acceptable salt.

These salts are those commonly used in pharmacy, such as acetate, benzoate, fumarate, maleate, citrate, tartrate, gentisate, methanesulfonate, ethanesulfonate, benzenesulfonate, laurylsulfonate, dobesilate and paratoluenesulfonate.

Subsequently, the amounts of methionine and other amino acids, such as cysteine and phenylalanine, are expressed in equivalents thereof in free form, non-salified, which may be in DL or L isomeric form.

Advantageously, the compositions of the invention comprise L-methionine. L-cysteine and L-phenylalanine in a molar ratio (L-methionine/L-cysteine/L-phenylalanine) comprised between 20/1/0.5 and 20/0.2/1 or preferably when the methionine/cysteine ratio is between 2 and 50 maintaining constant phenylalanine or mre preferably in the ratio (cysteine/phenylalanine) between 10 and 1, maintaining methionine constant and at maximum proportion.

The associations, according to the invention, may be used in daily dosages of 270 mg or between 150 a 270 mg of the sum of the three active ingredients in form of pure amino acid, such as inner salt, maintaining the indicated proportions of each one.

In human beings, the daily dosage may be constant at any age over 18 years old, to any individual to be treated or the type of healing or prophylactic treatment.

In the pharmaceutical compositions of the present invention, the active ingredients are generally formulated in dosage units containg 100 to 300 mg of active ingredients per dosage unit, maintaining the indicated proportions between the active ingredients.

The present invention has, therefore, as an object the pharmaceutical compositions containing, as active ingredient, an association of methionine, cysteine and phenylalanine. These compositions are, preferably, manufactured so as to be able to be orally or rectally administrated.

In the pharmaceutical compositions of the present invention for oral, intramuscular, intravenous, transdermal, local or rectal administration, the active ingredient may be administered in administration unit forms, mixed with traditional pharmaceutical supports, in animals and human beings. The suitable administration unit forms comprise the forms by oral route, such as tablets, gels, powders, granules and oral solutions or suspensions, the forms of sublingual and bucal administration, the intramuscular, intravenous administration forms, and the rectal administration forms.

When a solid composition is prepared in tablet form, the main active ingredient is mixed with the pharmaceutical vehicle, such as gelatine, starch, lactose, magnesium stearate, talc, gum arabic and the like. The tablets may be covered with sucrose or in another suitable fashion or they may also be treated in such a way that results in a long-lasting or delayed activity so that they continously release a predetermined amount of active ingredients.

A gel preparation may also be obtained by mixing the active ingredient having a dilluent and pouring the obtained mixture in hard or soft gels.

A syrup or elixir preparation may contain the active ingredient together with a sweetener, preferably non-caloric, methylparaben and propylparaben, as an antiseptic, as well as a flavoring agent and a suitable colorant.

Water-dispersible powders or granules may contain the active ingredient mixed with dispersing agents or umectant agents, or suspending agents, such as polyvinylpyrrolidone, in the same fashion as sweeteners or flavorants.

For rectal administration, suppositories are used which are prepared with ligands that conveniently melt at rectal temperature, for example cocoa butter, fatty acid esters having glycerin or polyethylene glycols.

For parenteral administration, aquous suspensions, isotonic saline solutions or injectable and sterile solutions are used, containing pharmaceutically compatible dispersing agents and/ or ume ctant agents, such as for example p ropylene glycol or butileneglycol.

The active ingredient may be equally formulated in the form of microcapsules, eventually having one or various supports or additives.

The active ingredients of the associations may be equally presented in complex form having a cyclodextrin, for example α, β or γ cyclodextrin, 2-hydroxypropyl-β-cyclodextrin ou methyl-β-cyclodextrin.

When the compositions of the invention are parenterally and/or orally administered to man, it is preferable that the daily dosage of methionine is comprised between 10 and 300 mg, the daily dosage of cysteine between 5 and 20 mg and the daily dosage of phenylalanine between 10 and 30 mg.

It should be noticed that, according to the invention, methionine, cysteine and phenylalanine may be administered by oral route, or the three of them by parenteral route or two of them by oral or parenteral route (cysteine and pheny)alanine) and the third, preferably methionine, by rectal route.

According to a preferred embodiment, the daily dosage of L-methionine to humans by parenteral, anal and/or oral route is comprised between 100 and 300 mg, preferably between 150 and 250 mg, the daily dosage de L-cysteine administered by parenteral, rectal and/or oral route being of 10 to 30 mg and the dosage of L-phenylalanine by parenteral, rectal and/or oral route between 5 and 30 mg or more preferably between 10 and 20mg.

Preferably, the dose of L-methionine is, in this case, 170 mg per day and the L-cysteine and L-phenylalanine dosages 20 and 10 mg respectively.

The associations of active ingredients, according to the invention, constituted the object of pharmacological studies. Tests were made and the results thereof were clinically observed. For such, various volunteers were randomly selected. However, in order to better control the tests, use was made of the classification system for hemorrhoids of broader use (Hardy et al., Dig. Surg, 2005, 22, 26-33) known as Goligher's classification, wherein hemorrhoids are classified in four different types.

In short, volunteers, aging from 20 to 80 years old, were tested and observed for 28 days by rectally administering suppositories of 2 g each, containing the active ingredients in the indicated proportion: methionine (170 mg/day), cysteine (20 mg/day) and phenylalanine (10 mg/day) each.

The results shown in Table 1 clearly indicate that the active ingredients of the present invention, when rectally administered for 3 to 4 days, inhibit the bleeding caused by hemorrhoids and in a long-lasting manner.

In all the cases, the joint administration of methionine, cysteine and phenylalanine resulted in a significant synergic effect resulting in the lessening of pain and discomfort of rectal bleedings caused by oxidative stress and/or by hemorrhoids of various types (Goligher type I, II, III and IV).

In comparison to simple additive effect observed between the anti-hemorrhagic effect of cysteine or of phenylalanine, this long-lasting regenerating activity of the intestine wall with anti-hemorrhagic effect is completely new and unexpected.

In the same way, this anti-hemorrhagic activity of the present association was potencialized by the association described herein resulting in completely new and unexpected improved treatment efficacy and long-lasting effect.

TABLE 1
Effect of isolated products and/or in association comparing the 4 different
types of hemorrhoids, following Goligher's classification, in adult human
beings and of both genders, ranging from 20 to 80 years old.

| Active | Dosages | | Goligher | Bleeding inhibition | Relapse after 5 |
|---|---|---|---|---|---|
| ingredients | (daily) | | (Type) | after 72 h (%) | years (%) |
| L-methionine | 180 | mg | I to IV | 70 ± 7% | 15 ± 2% |
| L-cysteine | 10 | mg | I to IV | 10 ± 1% | 80 ± 8% |
| L-phenylalanine | 10 | mg | I to IV | 2 ± 1% | 90 ± 9% |
| L-cysteine + | (10 ± 10) | mg | I to IV | 15 ± 2% | 70 ± 7% |
| L-phenylalanine | | | | | |
| L-methionine + | (180 ± 10) | mg | I to IV | 80 ± 8% | 10 ± 1% |
| L-phenylalanine | | | | | |
| L-methionine + | (180 ± 10) | mg | I to IV | 85 ± 8% | 10 ± 1% |
| L-cysteine | | | | | |
| L-methionine + | (180 ± 10 ± 10) | mg | I to IV | 100 ± 10% | 0% |
| L-cysteine + | | | | | |
| L-phenylalanine | | | | | |

The indicated values in the Table are average values in 20 experiments±standard errors (n=20).

In all the tested cases, no side effect was observed and/or reported by the volunteers treated, during or after the testing period.

In the same comparative period, untreated people present various episodes of hemorrhoidal crisis, and in all the treated cases herein by administering the present association, there was no relapse of pain and/or bleedings caused by hemorrhoids in the 5-year observation period after treatment, demonstrating the efficacy of the treatment with the present association as a new and unexpected fact.

1. Pharmaceutical composition for treating oxidative stress induced pathology, characterized in that it comprises:
an association of the active ingredient methionine having at least one pharmaceutical excipient;
the active ingredient being present in free state, such as inner salt, or in the form of pharmaceutically acceptable salt;
and said pharmaceutical composition being used for treating one of the oxidative stress induced pathologies, including intestinal system disorders, such as intestinal wall irritations, hemorrhoids of types 1, 2, 3 and 4 of Goligher classification.

2. Pharmaceutical composition, according to claim 1, characterized in that it comprises:
an association of the active ingredients cysteine and phenylalanine having at least one pharmaceutical excipient;
said active ingredients being present in a methionine/phenylalanine molar ratio between 2.5 and 12.5; and in a methionine/cysteine molar ratio between 2 and 10.5; and the three active ingredients being present in free state, such as inner salt, or in the form of pharmaceutically acceptable salt.

3. Pharmaceutical composition, according to claim 2, characterized in that the methionine/phenylalanine molar ratio is, preferably, between 3 and 9 and the methionine/cysteine molar ratio is, preferably, between 2 and 9.

4. Pharmaceutical composition, according to claim 1, characterized in that it is presented in a form administrable by parenteral, oral or rectal route.

5. Method for treating oxidative stress in a patient in need thereof which comprises administering a therapeutically effective dose of a composition as claimed in claim 1.

6. Method for treating oxidative stress induced pathology, characterized in that it comprises administering, to an individual, the composition as defined in claim 1, in dosages of 10 to 300 mg per day of methionine and of 5 to 20 mg per day of cysteine and of 10 to 30 mg per day of phenylalanine, the dosages being expressed in equivalent amount of methionine, cysteine and phenylalanine in the free form, of inner salt,

7. Method, according to claim 6, characterized in that it implies parenterally and/or orally and/or rectally administering 100 to 300 mg of methionine per day, 10 to 30 mg per day of cysteine and 5 to 30 mg of phenylalanine.

8. Method, according to claim 7, characterized in that it implies parenterally and/or orally and/or rectally administering 150 to 250 mg of methionine per day and 10 to 20 mg of phenylalanine

9. Method, according to claim 6, characterized in that it implies parenterally and/or orally and/or rectally administering 170 mg of methionine per day, 20 mg of cysteine per day and 10 mg per day of phenylalanine.

10. Method, according to claim 6, characterized in that methionine, cysteine and phenylalanine are administered by oral route, or the three of them by parenteral route or two, preferably cysteine and phenylalanine, by oral or parenteral route and the third, preferably methionine, by rectal route.
